(19) United States
(12) Patent Application Publication
(10) Pub. No.: US 2024/0355448 A1
(43) Pub. Date: Oct. 24, 2024

(54) PRECISION FUNCTIONAL MAPPING (PFM) TO PERSONALIZE NEUROMODULATION IN PSYCHIATRIC AND NEUROLOGICAL APPLICATIONS

(71) Applicant: Regents of the University of Minnesota, Minneapolis, MN (US)

(72) Inventors: Ziad Nahas, Minneapolis, MN (US); Damien A. Fair, Minneapolis, MN (US); David Darrow, Minneapolis, MN (US); Theoden Netoff, Minneapolis, MN (US); Robert Hermosillo, Minneapolis, MN (US)

(21) Appl. No.: 18/642,224

(22) Filed: Apr. 22, 2024

Related U.S. Application Data
(60) Provisional application No. 63/497,432, filed on Apr. 20, 2023.

(51) Int. Cl. G16H 20/30; G16H 15/00

(52) U.S. Cl. CPC G16H 20/30; G16H 15/00

(57) ABSTRACT

Precision functional mapping (PFM) is used to inform the planning, guidance, and/or monitoring of neuromodulation, including non-invasive brain stimulation, deep brain stimulation, prefrontal cortical stimulation, intracranial electrical stimulation, focused ultrasound-based neuromodulation, pharmacological-based neuromodulation, or the like.

### BACKGROUND

Since the wide-spread adoption of non-invasive brain stimulation such as transcranial direct current/alternating-current stimulation (“TDCS/TACS”) and transcranial magnetic stimulation (“TMS”), technologies for non-invasive brain stimulation have quickly outpaced techniques for neuronavigation. In TMS, early studies relied on a fixed distance from the motor cortex, which was then followed by an electroencephalography (“EEG”) cap positioning system. More recently, the gold standard for TMS uses real-time neuronavigation based on stereotactic image-guided positioning.

In clinical practice, most neuromodulation targeting is done by using relative anatomical distances. Unfortunately, this approach is insensible to individual differences in brain functional topography.

### SUMMARY OF THE DISCLOSURE

It is an aspect of the present disclosure to provide a method for planning, guiding, or monitoring a delivery of neuromodulation to a subject's brain. The method includes accessing precision functional mapping data with a computer system. One or more target locations are determined in the precision functional mapping data using the computer system, where the one or more target locations indicate locations to which neuromodulation should be delivered. Target localization data is generated by localizing the one or more target locations relative to the brain of the subject. The target localization data are then output with the computer system.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows that a point cloud of the pial surface is created (LEFT). A sphere depicts an example of the center of the desired placement of the stimulator. Each point is colored by a corresponding network.

FIG. 1B shows the regions that could be targeted by rotation of the stimulator remain colored while the others are uncolored.

FIG. 2A shows an example placement of the stimulator on the surface of the brain.

FIG. 2B shows an example of a stimulator paddle with 8 contact electrodes.

FIG. 3 is a flowchart of an example method for guiding or otherwise monitoring the delivery or administration of a neuromodulation therapy to a subject based on a precision functional mapping data.

FIG. 4 shows an example showing the placement of the stimulator on the surface of the brain, indicating which networks would be stimulated. Regions that would contact the stimulator are shown as a volume (cubes, or on the cortical surface). A reference Brodman area (Brodman area 10) is shown for reference.

FIG. 5 illustrates that presurgical planning can also be done using functional connectivity maps (i.e. correlation matrices). The paddles shown on the left represent an example configuration of anodes and cathodes that can be used during stimulation.

FIG. 6A shows an example of using the systems and methods described in the present disclosure to provide left DLPFC stimulation to improve attention in a subject.

FIG. 6B shows an example of using the systems and methods described in the present disclosure to provide salient and default network stimulation to induce a calming effect in a subject.

FIG. 6C shows an example of using the systems and methods described in the present disclosure to provide right frontopolar variant stimulation to improve mood in a subject.

FIG. 7 shows an example interpersonal variation in network organization or topography of three major networks: the Salience network (Sal), the default mode networks (DMN), and the frontoparietal network (FP). Standard Brodmann areas (BA 6 and BA10) are overlaid on the brain models as a reference.

FIG. 8 shows that adjacent regions within the brain can belong to distinct networks. In this example, the seeds region (showing whole-brain functional connectivity, represented as a red dot) starts in the salience network, but as the seed is moved inferiorly, the pattern of connectivity resembles the default mode network. Movement of the seed medially shows anti-correlation with the salience network. A stimulator device spans multiple networks, and each one can be separately targeted within the same stimulator paddle.

FIG. 9 shows an example in which when a standard parcellation is used (colors represent the boundaries of the Brodmann regions are shown), a clinician or investigator could mistakenly conclude that one homogenous region is being targeted.

FIG. 10 shows an example in which the boundary between networks is not necessarily sharp. In this example, a seed-based map that is placed within the salience network, is then moved inferiorly towards the frontoparietal network. A transition zone is observed that has shared features of connectivity of each of these networks.

FIGS. 11A-11D show that stimulation of regions of the brain can be used to targeted specific symptoms of a disorder. As an example, it is shown that the stimulation of distinct brain regions that target specific elements od depression. Stimulation of the location in FIG. 11A elicits feelings of joy, FIGS. 11B and 11C show stimulation that produces feelings of calmness, and FIG. 11D shows stimulation that increases attention and alertness.

FIG. 12 shows how stimulation settings (e.g., stimulation pulse frequency, or pulse width) can be adjusted using efficient optimization over time based on desired outcomes.

FIG. 13 is a block diagram of an example system for generating precision functional mapping (PFM) data and performing neuromodulation planning and/or guidance using the methods described in the present disclosure.

FIG. 14 is a block diagram of example components that can implement the system of FIG. 13.

### DETAILED DESCRIPTION

Described here are systems and methods for precision brain mapping (e.g., precision functional mapping (PFM)) informed intracranial electrode targeting to provide a reliable and individualized method for accessing dysfunctional brain networks to guide, plan, target, or otherwise monitor the delivery of neuromodulation therapies. For instance, neuromodulation therapies including non-invasive brain stimulation such as transcranial magnetic stimulation (TMS), transcranial direct current stimulation (TCDS), transcranial alternating current stimulation (TCAS), or the like, can be guided, planned, targeted, or otherwise monitored using the disclosed systems and methods. Additionally or alternatively, neuromodulation therapy can include other neurostimulations (e.g., deep brain stimulation, prefrontal cortical stimulation, intracranial electrical stimulation), focused ultrasound-based neuromodulation, pharmacological-based neuromodulation, or the like. The described systems and methods may also be used to enhance therapeutic efficacy of these neuromodulation therapies.

The disclosed systems and methods collect PFM data for precise mapping of the whole brain. The connectivity of the whole brain may be used to identify dysfunctional targets by looking for specific whole brain connectivity patterns, or by looking for specific whole brain networks of a given seed or seeds. That information may be leveraged for presurgical planning of the intracranial electrodes, or other neuromodulation delivery device.

In general, the systems and methods include obtaining PFM data for presurgical planning. Stimulation paddles and/or electrodes may then be modeled, designed, or otherwise selected (e.g., using Auto-CAD or the like) for presurgical planning. Additionally or alternatively, other neuromodulation delivery devices may be similarly modeled, designed, or otherwise selected. Dysfunctional networks of interest may then be presurgically identified based on patterns of functional connectivity (e.g., based on the PFM data). Additionally or alternatively, optimal treatment targets may also be identified based on patterns of functional connectivity based on patterns of functional connectivity (e.g., based on the PFM data). The identified targets may be exported into a neuronavigation system (e.g., a brain-MRI-compatible neuronavigation system) for stereotactic placement of the paddles or other electrodes. The placement of the paddles or electrodes may be confirmed with medical imaging, such as a CT scan of the subject.

Precision brain mapping of functional neural networks is a technique for examining individual network topography. Network topography across individuals appears to have some shared features, but the general shape and strength of connections remains highly individual-specific. It is an advantage of the systems and methods described in the present disclosure to generate PFM data for targeted brain stimulation or other neuromodulation therapy delivery. The inter-subject variability in the topography of functional networks limits the value of probabilistic targeting; thus, using PFM data as described in the present disclosure allows for individually and reliably identifying functional networks and optimal targets of interest to inform precise targeting for brain stimulation or other neuromodulation therapies.

It is another advantage of the systems and methods described in the present disclosure to also use PFM data to monitor and/or measure the efficacy of targeted brain stimulation or other neuromodulation therapies.

Presurgical planning is typically performed using anatomical landmarks of the cerebral cortex. Using the systems and methods described in the present disclosure, presurgical planning may be implemented based on the brain's patterns of functional activity. Using anatomical MRI scans and high resolution resting state functional connectivity, mapping is performed on the patient. Using this data, a 3D reconstructed model of the brain may be created. In addition, 3D models of cortical brain stimulators are created. Using a 3D model of the brain and 3D models of the neuromodulation device(s) (e.g., stimulators or electrodes), one can simulate placement of the cerebral cortex and predict which anatomical regions will be stimulated. For presurgical planning, placement of cortical electrodes can be based on the functional connectivity and neural network maps. The combination of multiple simulators can then be used to refine therapeutic interventions.

A high resolution anatomical MRI scan (e.g., a T1-weighted image) may be obtained along with resting state functional MRI (rs-fMRI). Timeseries data are created for voxels that depict blood-oxygen-level dependent (BOLD) signals. The BOLD data are projected to a surface reconstruction (mesh) of the cortex. Time course signals are created for each grayordinate. A grayordinate is a brainordinate within the gray matter of a subject's brain, and a brainordinate is a coordinate (e.g., a particular location) within a subject's brain. As one example, a brainordinate can be specified by a surface vertex, or node. As another example, a brainordinate can be specified by a volume voxel. Thus, a grayordinate corresponds to a particular location in the gray matter that can be specified as gray-matter surface vertices (e.g., cortical gray matter), gray-matter volume voxels (e.g., subcortical gray matter), or both.

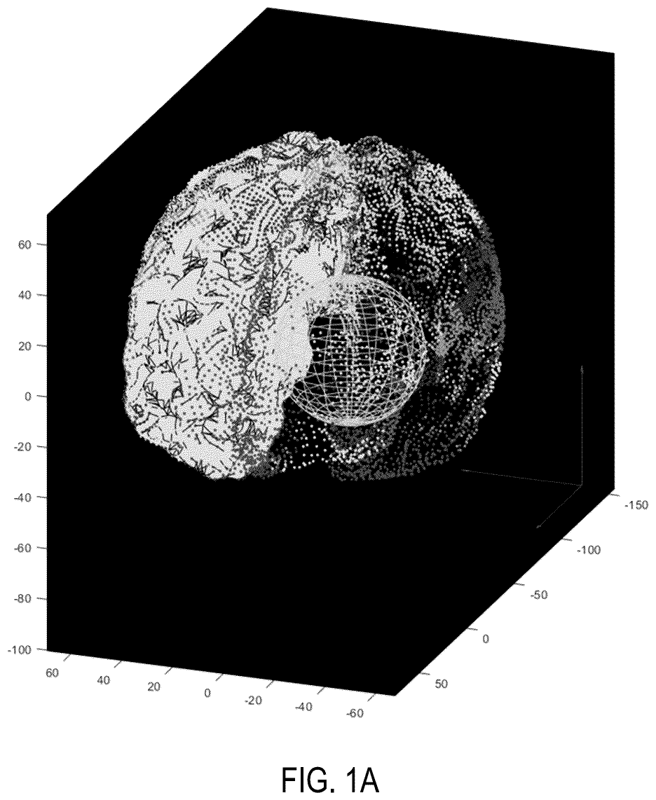

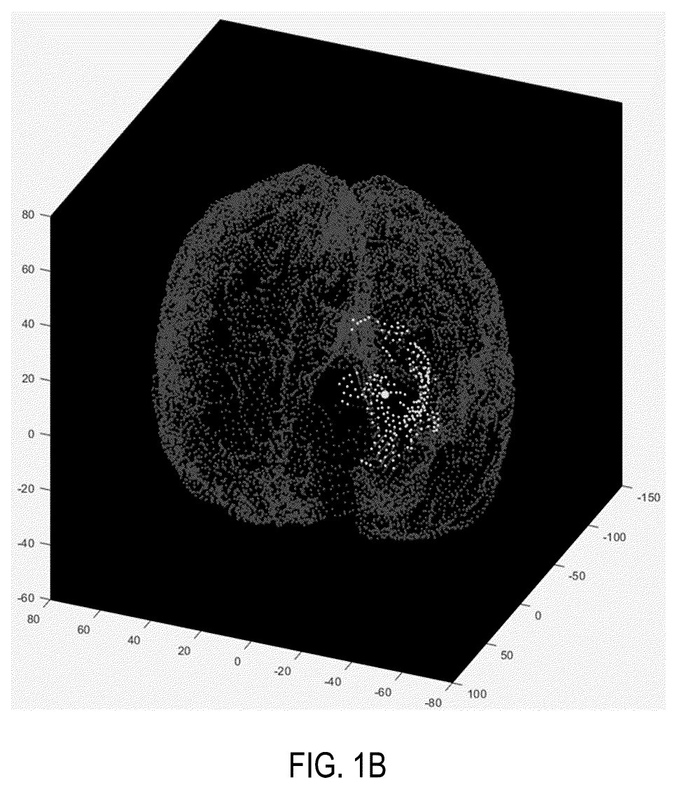

A 3D mesh of the brain's outer surface may then be created (e.g., using FreeSurfer software). The mesh of the outer surface may be transformed into a point cloud in space (FIGS. 1A and 1B). A correlation matrix may be computed from the time course signal data for each grayordinate. The correlation matrix data may then be used to perform precision functional mapping. There are a variety of techniques for generating PFM data, such as template matching and InfoMap. For example, the methods described in co-pending U.S. Patent Application Publication No. US 2023/0115330, which is herein incorporated by reference in its entirety, may be used to create PFM data in some examples.

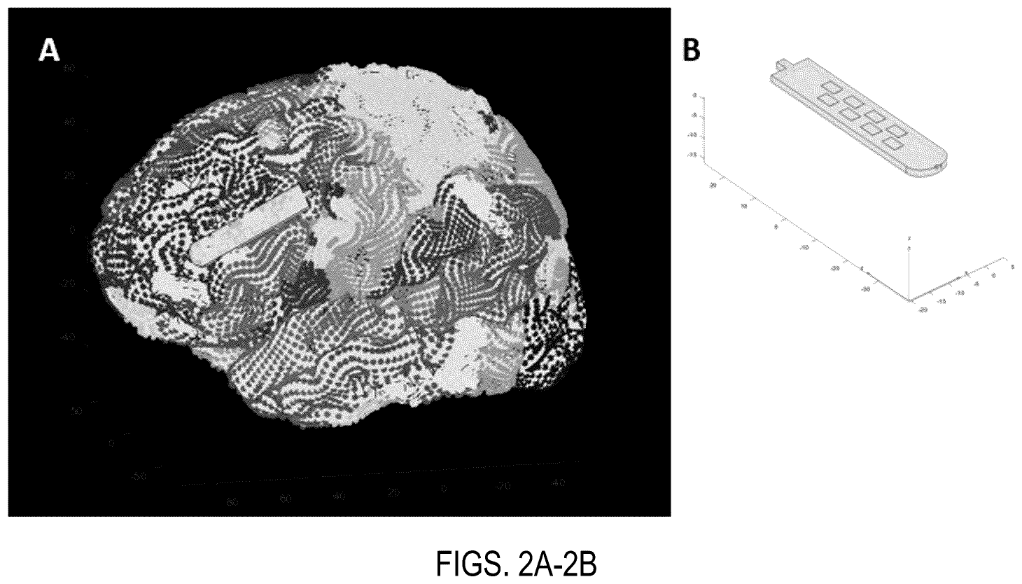

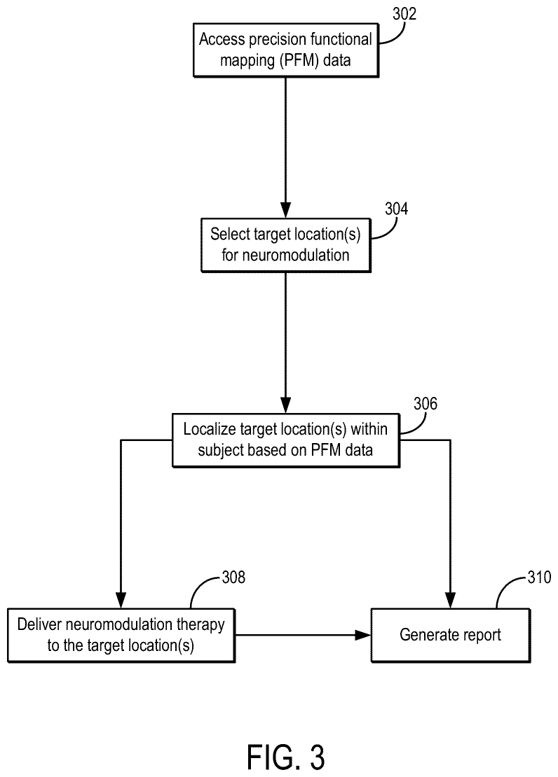

Stimulator 3D models may be created using Computer-Aided Drafting (CAD) software. The 3D model of the brain and the stimulator may be loaded into a virtual environment (FIGS. 2A and 2B). Using a map of the depth of the sulcus of the brain, one can determine which folds of the brain are the most external, allowing one to determine which brain regions are accessible for direct cortical stimulation. Once the user provides a desired stimulator placement, the software may calculate a plane that is tangential to the cortical surface. The software may then model a region of influence around each electrode of the stimulator panel, and may predict which brain regions/network will be stimulated. The software may output a placement map that can then be uploaded to a neuronavigation system.

The efficacy of the stimulation can then be monitored by selective stimulation of electrodes. Furthermore, multiple stimulators can be used to achieve the desired combination of symptoms/behaviors that best correspond with patient outcomes.

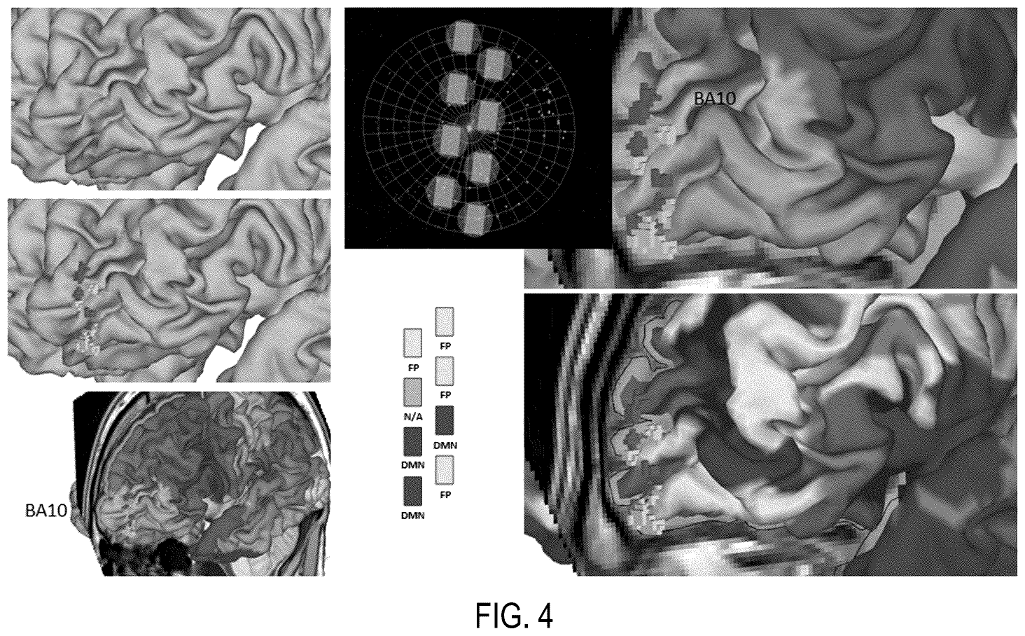

Referring now to FIG. 3, a flowchart is illustrated as setting forth the steps of an example method for guiding or otherwise monitoring the delivery or administration of a neuromodulation therapy to a subject based on a precision functional mapping data.

The method includes accessing a PFM data with a computer system, as indicated at step 302. Accessing the PFM data can include retrieving previously generated data from a memory or other data storage device or medium. Additionally or alternatively, accessing the PFM data can include generating the data with the computer system, such as by generating PFM data from magnetic resonance data and/or time course signal data using the methods described in the present disclosure.

As a non-limiting example, precision brain mapping of functional neural networks is a technique for examining individual network topography. Network topography across individuals appears to have some shared features, but the general shape and strength of connections remains highly individual-specific. PFM data may include individual-specific network maps. As one example, the PFM data may include an individual-specific functional network map and/or an individual-specific integration zone map.

A functional network map can be generated using an overlapping template matching technique, or another suitable technique. In an overlapping template matching technique, a functional network map can be generated from functional network template data corresponding to a plurality of different functional networks. The functional network template data can include templates for the following functional networks: the default mode network (DMN), the visual network (VIS), the frontal parietal network (FPN), the dorsal attention network (DAN), the ventral attention network (VAN), the salience network (Sal), the cingulo-opercular network (CO), the sensorimotor dorsal network (SMD), the sensorimotor lateral network (SML), the auditory network (AUD), the temporal pole network (Tpole), the medial temporal network (MTL), the parietal occipital network (PON), and the parietal medial network (PMN). Sensory and motor systems can be combined due to the coupled nature of activation. In other implementations, the templates can include fewer of these functional networks and/or can include additional functional networks. From the functional network template data, individual-specific network assignments can be determined. A voxelwise correlation matrix can be generated by correlating the BOLD signals for each grayordinate with the BOLD signals every other grayordinate represented in time course signal data acquired from the subject. The similarity between the correlation matrix and one or more of the templates in the functional network template data is then computed. Based on the similarity values, each grayordinate is assigned to one or more functional networks, generating output as one or more individual-specific functional network maps.

An integration zone map can be generated by determining the functional networks associated with each grayordinate in an individual-specific functional network map and assigning grayordinates in the integration zone map to one or more integration zones based on the functional networks associated with each grayordinate. As one example, the number of functional network assignments at each grayordinate can be counted, and the counts of overlapping functional networks at each grayordinate can be stored as the integration zone map. The count of networks at each grayordinate can define an integration zone. Additionally or alternatively, the specific functional networks assigned to each grayordinate can also be stored for each grayordinate in the integration zone map. In some embodiments, the number of networks and specific networks at each grayordinate can collectively define different integration zones. For example, clusters of grayordinates associated with overlapping functional networks can be identified and integration zones can be defined based on those identified clusters.

Based on the PFM, one or more target locations to which neuromodulation therapy will be delivered are selected using the computer system, as indicated at step 304. The one or more target locations can be grayordinates or other brainordinates that are selected based on the condition for which neuromodulation therapy is being provided. Additionally or alternatively, a target location can include a group or cluster of grayordinates, such as a group or cluster of grayordinates associated with a common functional network, two or more different functional networks, or the like.

Thus, in general, selecting a target location for neuromodulation therapy can include determining a functional network, or networks, that when modulated by a neuromodulation therapy would provide a therapeutic effect to the subject (e.g., by controlling and/or improving the condition of the subject, or the like). As one example, target locations can be selected based on the condition to be treated. In some instances, selecting target locations can include selecting locations in the PFM data that have a probability of being assigned to a particular functional network that is above a certain threshold (e.g., 50%, 60%, 70%, 80%, 90%, etc.).

After the one or more target locations (e.g., grayordinates) to receive neuromodulation therapy are selected based on the PFM data, the target locations are localized within the subject, as indicated at step 306, thereby generating target localization data indicating the one or more target locations for receiving neuromodulation. For example, localizing the target locations can include identifying the target locations relative to the subject's anatomy, such that neuromodulation therapy can be delivered to the anatomical locations within the subject that correspond to the target locations selected relative to the PFM data.

In a non-limiting example, the target locations can be localized within the subject by accessing medical image data of the subject using the computer system, where the medical image data include, for example, at least one anatomical image depicting the brain of the subject to whom neuromodulation treatment will be delivered. As an example, the medical image data may be anatomical magnetic resonance images of the subject. Accessing such magnetic resonance images can include retrieving previously acquired images from a memory or other data storage device or medium. Additionally or alternatively, accessing the magnetic resonance images can include acquiring the images with an MRI system and transferring or otherwise communicating the data to the computer system, which in some embodiments may be a part of the MRI system.

The medical image data and the PFM data can then be coregistered, such that locations (e.g., grayordinates) in the PFM data can be associated with anatomical locations within the subject. In this way, the target locations selected in the PFM data can be localized relative to the subject's own anatomy.

In some embodiments, the neuromodulation device can be modeled and coregistered with the PFM data and/or other medical image data. For instance, the electrodes of a neurostimulation device can be modeled and their position and orientation determined based on the model. The locations of the electrodes can then be superimposed on the PFM data and/or other medical image data to create composite image data of the medical images, PFM data, and targets of interest. The composite image data can be exported to the neuromodulation device to assist with delivery of the neuromodulation.

Neuromodulation therapy is then delivered to the localized target location(s). For example, neuromodulation therapies such as TMS, TDCS, TACS, focused ultrasound, or other suitable neuromodulation, can be delivered transcranially to the localized target location(s) by positioning the neurostimulation device relative to the localized target locations, as indicated at step 308. Similarly, neuromodulation therapies such as deep brain stimulation can be delivered by implanting an electrode adjacent the localized target locations, or otherwise directing electrical stimulation to the localized target locations.

In some embodiments, the neuromodulation delivered can be adjusted based on the particular localized target location. For example, if a localized target location corresponds to a grayordinate associated with an integration zone where multiple functional networks interact, a different neuromodulation may be delivered to that grayordinate than to a grayordinate associated with only a single functional network. In this way, the systems and methods described in the present disclosure can provide adaptive neuromodulation therapy that reduces or otherwise avoids modulating or overmodulating functional networks that may not provide a therapeutic effect to the subject.

Additionally or alternatively, a report can be generated by the computer system based on the localized target locations, as indicated at step 310. The report can indicate a neuromodulation treatment plan for delivering the neuromodulation to the one or more target locations. The treatment plan can include one or more neuromodulation settings for controlling the operation of the neuromodulation device, such as the settings that may be delivered in step 308. Additionally or alternatively, the report can include images (e.g., the medical images of the subject, the PFM data) and can provide image guidance for delivering neuromodulation to the localized target locations. In still other example, the report can include data reporting on the delivery of neuromodulation to the subject, including neural activity measured in response to the delivered neuromodulation, subject feedback on preference for one or more selections of neuromodulation settings, and so on.

As described below, in some instances the neuromodulation settings can be updated based on feedback data received from the subject and/or the neuromodulation device. For example, feedback data indicating the subject's preference for one or more neuromodulation settings can be received and processed using a Bayesian optimization framework to generate updated neuromodulation settings. Any other suitable form of closed-loop feedback may also be used to update the neuromodulation settings.

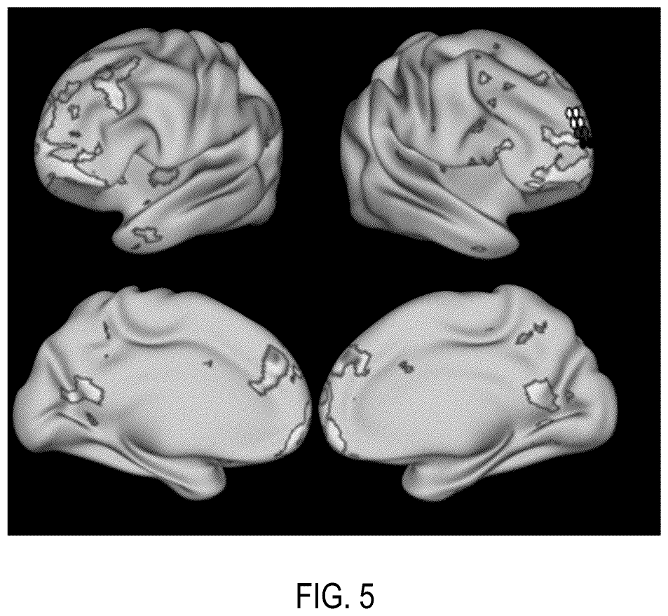

FIG. 4 illustrates an example showing the placement of a neuromodulation device (e.g., a neurostimulation paddle) on the surface of the brain, indicating which networks would be stimulated based on the localized targe locations determined using the methods described in the present disclosure. Regions that would contact the stimulator are shown as a volume (cubes, or on the cortical surface). A reference Brodman area (Brodman area 10) is shown for reference. These data may be stored in a generated report that can displayed or otherwise output to a user.

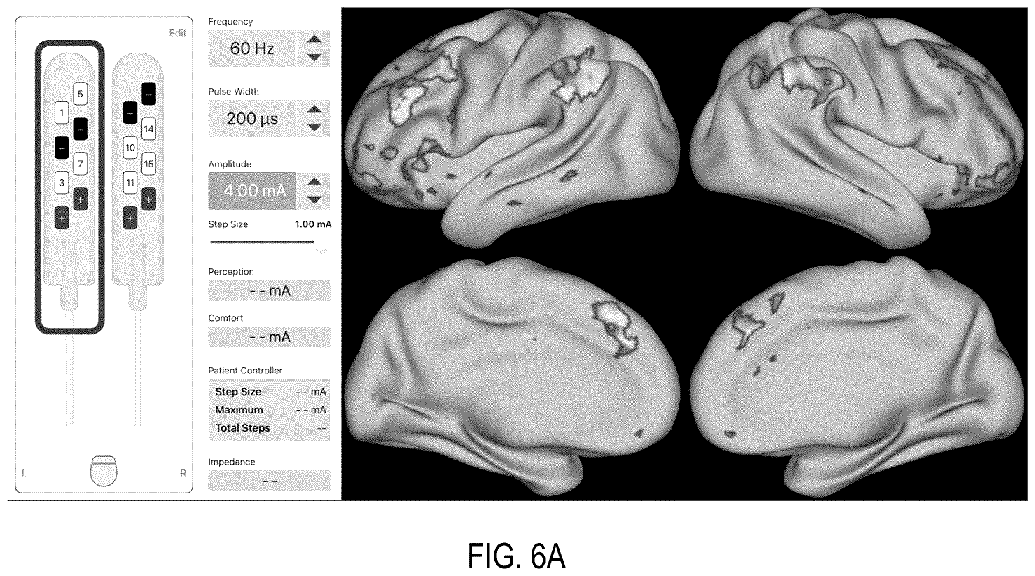

In some implementations, the PFM data may additionally or alternatively includes functional connectivity maps (e.g., correlation matrices). FIG. 5 illustrates an example report that may be generated for presurgical planning using such functional connectivity maps. Localized target locations can be seen overlaid on the upper righthand functional connectivity map.

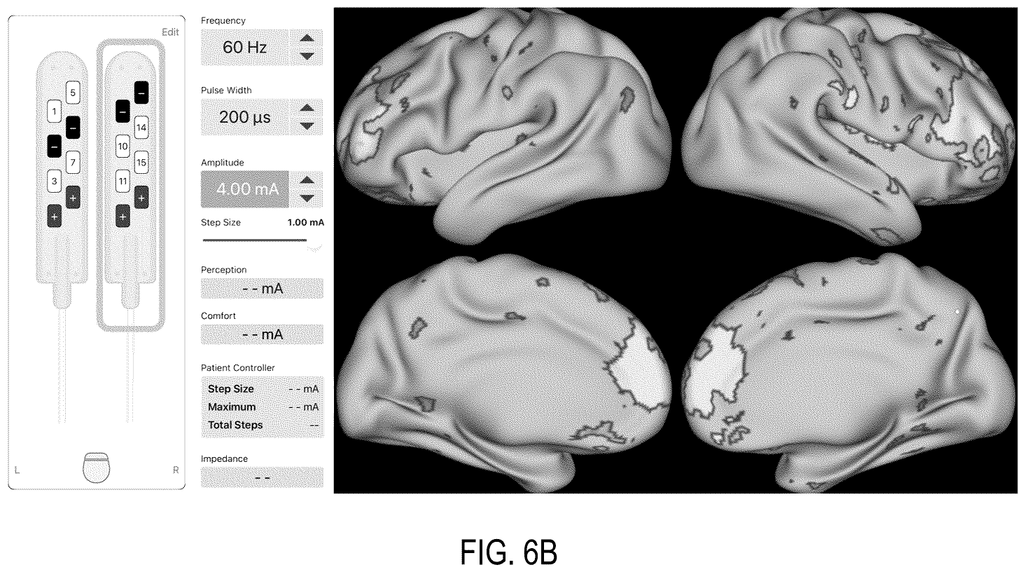

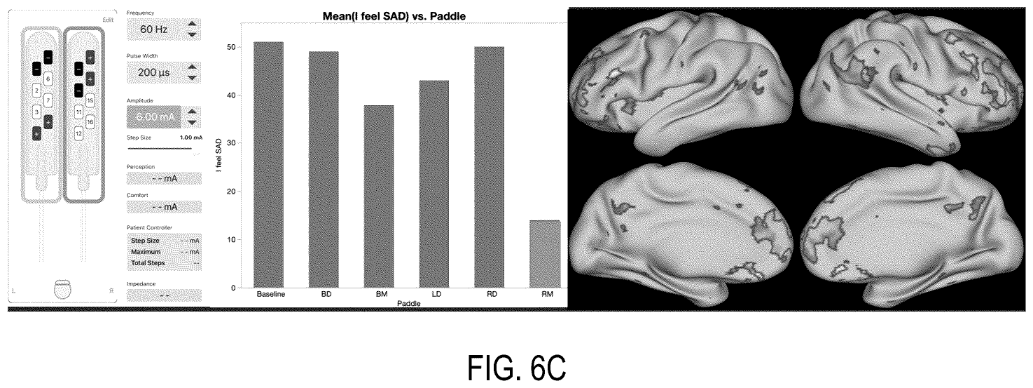

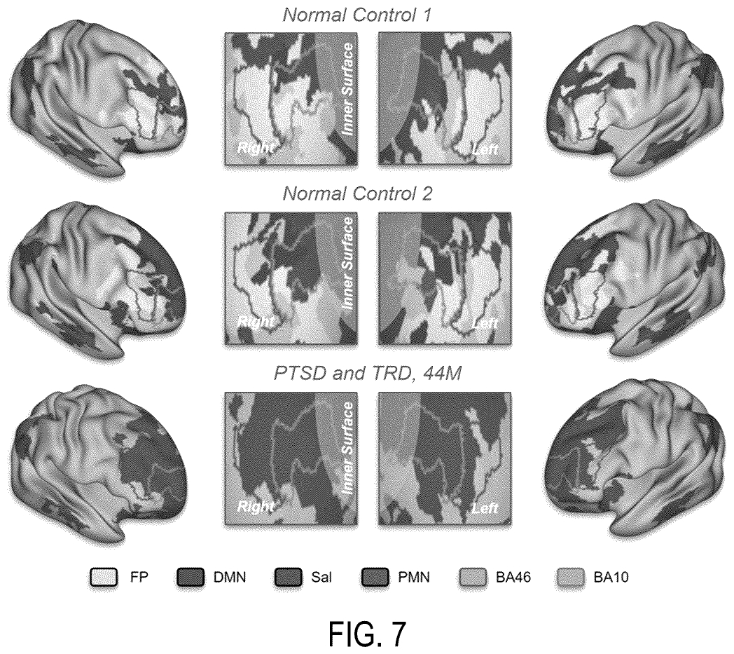

Although the examples provided above are described with respect to determining locations for delivering neuromodulation therapy to treat depression, other neurological conditions can also be treated using the systems and methods described in the present disclosure. For example, FIG. 6A shows an example of using the systems and methods described in the present disclosure to provide left DLPFC stimulation to improve attention in a subject. FIG. 6B shows an example of using the systems and methods described in the present disclosure to provide salient and default network stimulation to induce a calming effect in a subject. FIG. 6C shows an example of using the systems and methods described in the present disclosure to provide right frontopolar variant stimulation to improve mood in a subject.

In an example study, the disclosed systems and methods were used to localize target locations for delivering neuromodulation to subject with severe treatment-resistant depression (sTRD). Precision imaging guided direct cortical stimulation was combined with Bayesian optimization to set stimulation parameters and provide a scalable therapy to acutely improve mood and attention and, consequently, alleviate the symptoms of sTRD. The PFM-based techniques described in the present disclosure were used to identify the default mode, salience, and frontoparietal networks at the individual level with sTRD. Stimulation paddles were then stereotactically implanted in subjects to access these networks. Postoperatively, Bayesian optimization was used to iteratively adjust the stimulation parameters. Full remission of symptoms was achieved within six months and maintained past twelve months.

In the example study, a unipolar TRD patient was selected. The patient met criteria for a severe depressive episode for either major depressive disorder according to DSM-V, with a current depressive episode of at least 2 years in duration. Subjects met criteria for antidepressant treatment history form (ATHF) severity ≥3 failed trials in the current depressive episode. The patient was implanted with bilateral PCS using Eterna™ devices and Lamitrode 44 (Abbott Laboratories) electrodes. Symptom improvement was targeted in a series of programming steps and on a prescribed schedule for six months. Optimal settings were then locked in month seven through twelve. Detailed measures of symptoms and behavior were recorded regularly. Additional machine learning algorithms explored key electrophysiological and behavioral markers that can best predict antidepressant response.

Prior to surgery, subject underwent a high-resolution structural MRI scan with and without contrast to identify anatomical landmarks for rostral anterior and lateral prefrontal cortex. Participants also underwent a PFM scan (see scanning parameters below). For pre-surgical planning, a template with anatomically delineated cytoarchitecturally defined areas was morphed and co-registered onto the structural scan. Similarly, the PFM data were also morphed and co-registered onto the structural scan. The boundaries of dysfunctional networks of interest (e.g., default, salience, and fronto-parietal) and optimal treatment targets were then identified using the methods described in the present disclosure based on connectivity with the subgenual cortex and electrode coverage of multiple brain networks. The Lamitrode 44 stimulation paddles were modeled in Auto-CAD and the optimal point of entry (burr hole) and trajectory for the paddle insertion was determined. Once the position and orientation of the electrodes was determined, the location of the eight electrodes on each of the four paddles was superimposed on the patient's native structural scan with the model simulation as highlighted in preliminary studies. The composite of the structural MRI, PFM and targets of interest was then exported into a Medtronic Stealth Neuronavigation (Medtronic, Dublin, Ireland) for stereotactic placement of the electrodes. Surgical planning took place one week prior to surgery.

PCS was implanted in two stages separated by four days of ICU monitoring and stimulation optimization. In the first surgery, 3D tagged MRI image data were imported into Medtronic Stealth Neuronavigation (Medtronic, Dublin, Ireland). For stereotactic placement of each Lamitrode 44 paddle lead, targeted cortical areas were mapped onto the scalp using a pointer wand with the linear “look ahead” function activated to indicate the underlying cortical area of interest. A coronal incision was done behind the hairline. Co-registration with intraoperative CT scan and neuronavigation equipment allowed adjustment or verification of accurate subdural lead placements. The paddle leads were anchored to the edge of the craniectomy slit openings. Lead extensions allowed externalization for intracranial recordings stimulation testing in the ICU. In the second surgery, the participant was brought back to the operating room four days later and an Eterna IPG (Abbott Laboratories) with two channels was implanted on each side in the chest wall. One device was connected to both paddles over the anterior frontal poles. The second device was connected to both paddles over the mid-lateral prefrontal cortices. Channel 1 and 2 control the right and left leads, respectively. Postoperative CT images confirmed that the final position of the electrode matched the pre-surgical desired position of the electrode by co-registering the CT to the preoperative MRI.

For image acquisition, high resolution whole-brain anatomical T1-weighted (MP-RAGE) and T2-weighted (SPACE) scans were performed using scan parameters matched to an HCP-lifespan protocol. Scan parameters were: T1: TE/TI/TR=3.65/1100/2530 ms, flip angle=7 degrees, voxel size=0.8 mm isotropic, scan time≈7 min. T2: TE/TR=564 ms/3200 ms, variable flip angle, voxel size=0.8 mm isotropic, scan time≈6 min. The MRI scan was advantageous for stereotactic surgical placement of PCS leads.

Resting-state fMRI (rs-fMRI) data were collected using a multiband multi-echo (MB-ME) sequence with the following parameters: echo times (TE)=14.2, 38.93, 63.66, 88.39 ms; repetition time (TR)=1761 ms; MB factor=6; partial Fourier fraction=0.75; GRAPPA factor=2; flip angle=68 degrees; 72 oblique axial 2 mm thick slices; field of view (FOV)=220 mm; 2×2 mm in-plane resolution (110×110 matrix size). The first three volumes per run were discarded from data analysis to ensure that magnetization reached steady state. The last three volumes per run were acquired without RF excitation in order to estimate thermal noise level for NORDIC denoising and were discarded after NORDIC was applied. Participants were asked to look directly at the center of a fixation cross throughout a run or to freely view an animated video throughout a run. Stimulus types were alternated throughout a session. A scanning session had up to four resting-state runs of 550 volumes (16 minutes) each, for a total of up to 64 minutes in a session.

Data were collected using a 32 channel head coil and HCP scan parameters. The collected data were preprocessed using the HCP minimal preprocessing pipeline. Structural image (e.g., T1-weighted image and T2-weighted image) processing included FreeSurfer segmentation and parcellation, calculation of cortical myelin maps, and registration to CIFTI space. fMRI preprocessing included motion and distortion correction; registering fMRI to structural data and CIFTI space; artifact detection and removal using FSL-FIX; as well as improved surface registration using MSMALL. Further preprocessing (CSF and white and gray matter regression, frame censoring, and band pass filtering) followed by individual precision functional network mapping were carried out on the preprocessed data.

Individual-level functional connectivity analyses included extracting mean time courses from all grayordinates/voxels. The extracted time course data were made into correlation matrices of the whole brain using low-motion frames. The whole-brain correlation matrices were used as the input for a community detection method to identify individual-specific neural networks using either a supervised (template matching) or unsupervised approach (Infomap). Both of these methods were used to assign network labels to each grayordinate. The regional part of each network of interest (e.g., the anterior portion of the DMN) were defined and leveraged for planning and targeting.

As described above, the community detection method can use a graph theory-based algorithm (e.g., Infomap), or can use a template matching technique. In this example study, an Infomap-based technique was used. Vertices/voxels within 30 mm of each other were set to zero in the matrix to avoid biasing network membership for nearby connections that had undergone spatial smoothing. The resulting correlation matrix was then thresholded at a range of density thresholds (0.3%, 0.4%, 0.5%, 1%, 1.5%, 2.0%, 2.5%, 3.0%) and each one was used as an input for Infomap. For instances where Infomap was implemented on combined cortical and subcortical data, the range of density thresholds was extended to include 4% and 5%. Infomap calculates the network assignment based on an optimized code length using a flow-based method. Networks that are computed in the group average are labeled based on similar patterns of activation observed in the scientific literature. Small networks with 400 or fewer gray ordinates were defined as “unassigned”.

Networks identified in each individual were then labeled based on the Jaccard Similarity to a network observed in the group average, however, often individuals will retain novel networks that are not observed in group averaging, and these remain unlabeled. The list of networks included are the default mode network (DMN), the visual network (VIS), the frontal parietal network (FPN), The premotor network (PMN), the dorsal attention network (DAN), the ventral attention network (VAN), the salience network (Sal), the cingulo-opercular network (CO), the sensorimotor dorsal network (SMD), the sensorimotor lateral Network (SML), the auditory network (AUD), the anterior medial temporal network (AMTL), the posterior medial temporal network (post MTL), parieto-occipital network PON, and the parietal medial network (PMN). In each subject and in the average, a “consensus” network assignment was determined across the various thresholds, by giving each node the assignment it had at the sparsest possible threshold at which it was successfully assigned to one of the known group networks. Contiguous network clusters that were smaller than 30 grayordinates were removed and merged into neighboring networks, with the largest networks given priority.

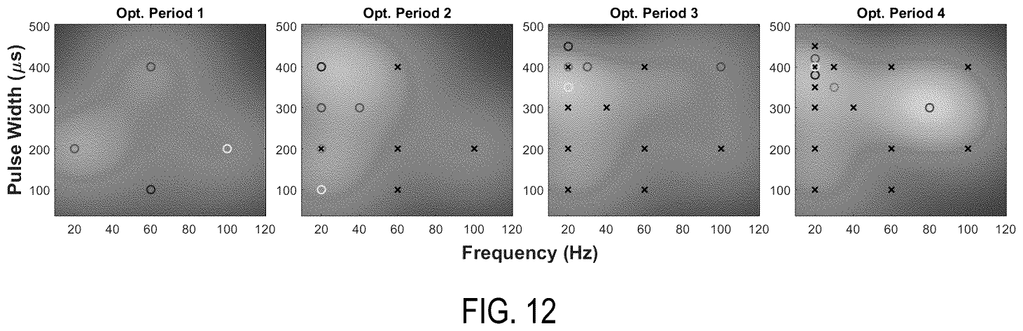

To optimize the stimulation parameters for the treatment of depression, Bayesian optimization was implemented. First, in an initial testing stage, the patient was provided with nine settings, each with different frequencies and pulse widths. The patient tested each setting at home for a period of approximately 4-5 days, carefully monitoring their depression scores, sleep duration, and ranking of each setting. Example sets of neuromodulation settings and corresponding user preferences are illustrated in FIG. 12. Next, in a scoring equation stage, using the collected data, a value for each setting was calculated based on the scoring equation:

$$v = DS + {β}_{1} \cdot SS + {β}_{2} \cdot RP ;$$

Here, DS represents the average depression scores, SS represents the average sleep duration, and RP signifies the rank of each setting. The β₁ and β₂ coefficients serve as weighting factors for these scores, allowing the effectiveness of each setting to be quantitatively evaluated.

Then, in a Gaussian process regression (GPR) stage, at the end of the month, a value surface was generated by fitting a GPR model to the collected data. The GPR estimates both the mean value, μ(f,p), and the standard deviation, σ(f,p), across the entire parameter space. In a neurostimulation setting selection stage, the next settings for testing were selected using either Thompson's sampling or SafeOpt. With Thompsons's sampling random surfaces are drawn from the GPR function and the setting which has the highest values is selected. This samples points near the maximum more often (exploitation), but also selects parameters in untested parameter space (exploration). SafeOpt is similar, but starts with a narrow parameter range around a known safe setting, and then expands the parameter range in areas that are expected to be better than the worst acceptable setting. Selected points are chosen so that they are not too close to each other and balance exploitation and exploration.

This process was then repeated until convergence. For instance, sampling and testing of settings were repeated, the response surface was revised, and new settings were selected until some measure of convergence was achieved.

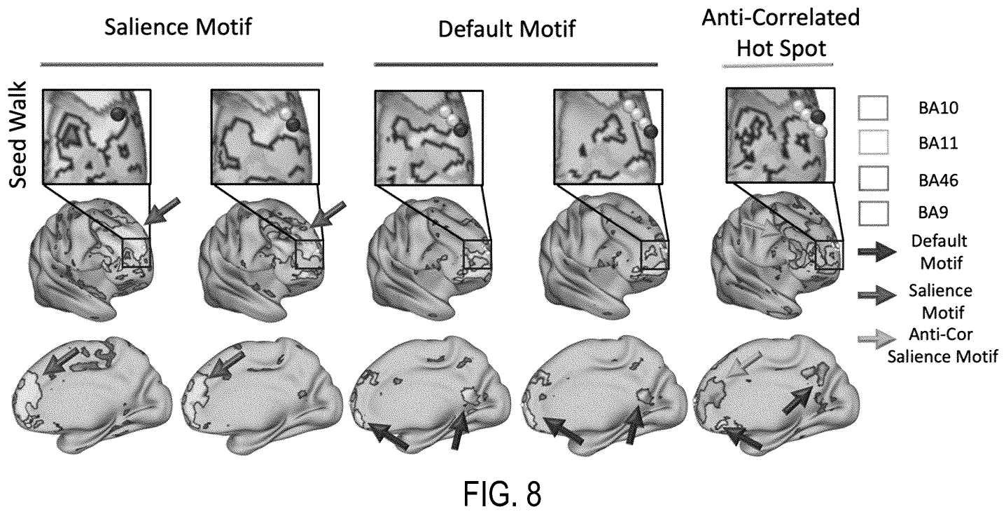

Subdural direct cortical stimulation, or precision cortical stimulation (PCS) combined with PFM and Bayesian optimization, was leveraged for a patient with TRD. Community detection was first utilized to characterize brain networks in the patient, leveraging PFM data acquisitions. It was observed that network patterns were highly atypical. In particular, BA10 and BA46 were primarily represented by the salience motif, which was overrepresented compared to other typically functioning adults (FIG. 7).

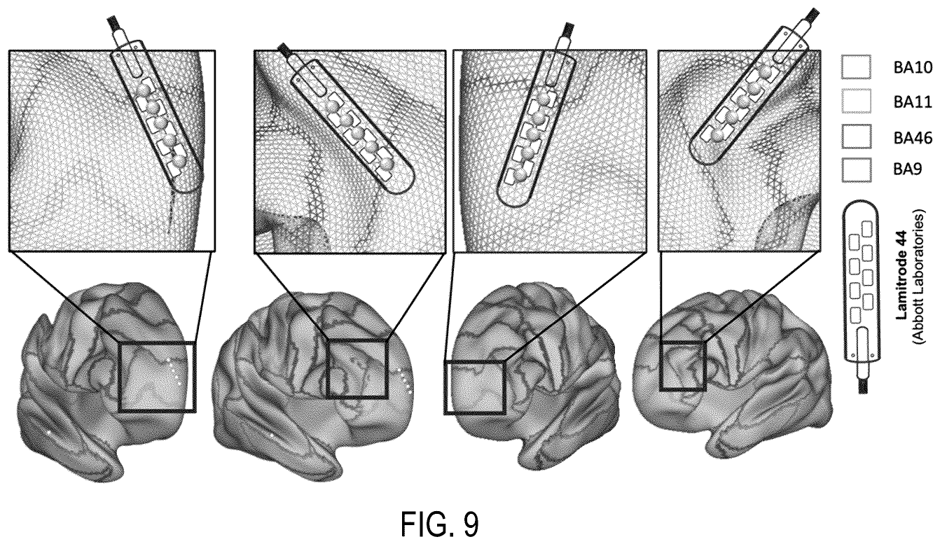

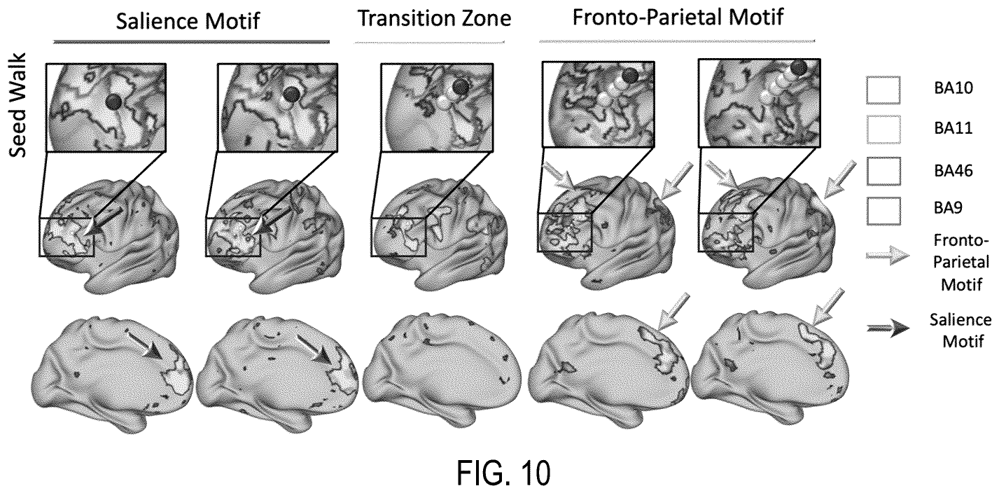

This initial examination provided the guidance to conduct a “seed walk” in BA10 to identify a clear transition from the atypical salience motif to the default network motif in the patient (FIG. 8). This transition is also remarkable for a uniquely negatively correlated variant (or hot spot) with the salience motif that is strongly associated with sub-colossal cortex (FIG. 8). This seed walk showed a clear transition from salience to default motifs and provided a marker for surgical planning and the placement of electrodes (FIG. 9).

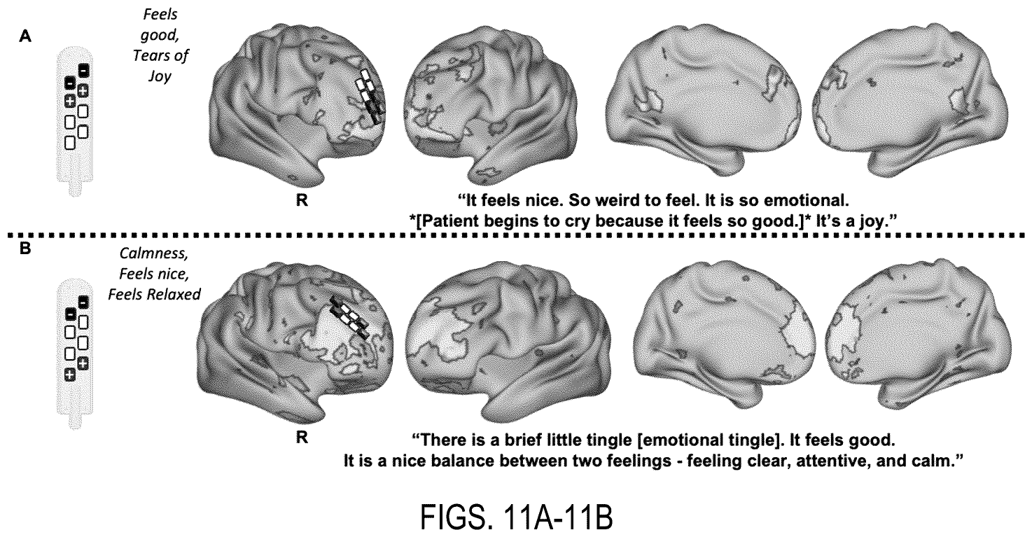

In the left hemisphere, the salience motif encompassed a large portion of BA10 (FIG. 7). However, a seed walk from the border of BA10 through BA46 to the border of BA9 showed a clear transition from the salience motif to the frontoparietal motif (FIG. 10). This trajectory became the target for surgical planning assuring contacts within the frontoparietal system in the patient (FIG. 10).

The unique seed walks within left and right BA10 and BA46 provided the necessary angle and orientations to optimally position the electrodes such that stimulation could occur within the salience, default, and frontoparietal motifs. This was accomplished by first modeling the Lamitrode 44 stimulation paddles in Auto-CAD. Paddles were simulated on the pial surface using in house software (Matlab) and positioned along the axes identified during the planning stage (FIG. 9). Each contact was then exported back to the native T1 pial surface, and finally to native T1 volume compatible with neuronavigation systems for stereotactic placement of the electrodes. An intra-operative CT scan confirmed the highly accurate targeting of the loci-of-interest.

In the following three post-operative days, various stimulation parameters were tested in the Neuro ICU along with recording the subjective changes in mood, anxiety, and attention. Via a patient masked sham-controlled parametric testing, reproducible effects of stimulation on the patient's mood and attention were observed. Order was randomized, with “no stimulation condition” (0 V) always occurring first and at least once more during testing.

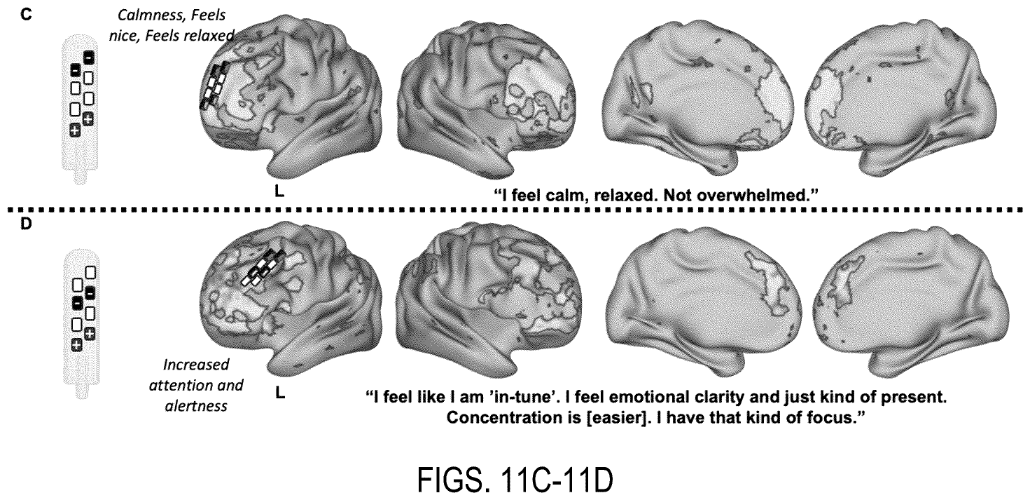

Stimulation paradigms resulted in immediate positive responses, which were compared against sham-controlled stimulation. The most salient effect was that of the default motifs (FIGS. 11A-11D). Here, the patient presented with overwhelming feelings of joy, leveraging the upper most contacts (Anodes: 10,14; Cathodes, 9,13) at a frequency of 60 hz, pulse width 200, and Amplitude of 6. Noting “It feels nice. So weird to feel. It is so emotional.” The patient started crying because it feels good. “It is a joy.” When the device was turned off, the pleasant sensation went down a little bit but it still feels good. It was repeated and the patient got the same feeling. This effect was not replicated when stimulating across all contacts. The effect was also replicated in the lab at two-week follow-up.

On average, stimulating any contact that involved the salience motif related to feeling calm and relaxed (FIGS. 11B, 11C). Stimulation of the frontoparietal motif resulted in clarity. The patient did not feel any sluggishness or word hesitation. It felt good in terms of emotions and presence. It was easier for the patient to focus (FIG. 11D).

Prior to discharge, the patient was implanted with two Proclaim Elite SCS systems (Abbott Laboratories) connected to the bilateral frontopolar and DLPFC paddles. Once discharged from the hospital, the patient returned to the lab after two weeks where testing and findings from the ICU were replicated. From there, the patient presented monthly to the lab and the stimulation parameters were optimized using the Bayesian techniques described above, which are based on an evenly weighted combination of the patient's subjective preference ranking (for five settings, 1-10 with 10 being the most preferred), amount of sleep in hours, and reported mood (1-100, 1 being severely depressed, 100 being severely manic, and 50 being stable). At each 1-month interval, the patient was pre-programed with 4 to 5 different settings, was kept blinded to the settings, and was asked to toggle between settings every week while rating their daily mood, hours of sleep, and rank ordered preferred programming options. From this, a response surface for improved depressive symptoms was generated. In this way, the most effective treatment was adaptively and efficiently selected, while accounting for possible neuronal adaptation to chronic stimulation.

Precision functional mapping (PFM) has been shown to enhance spatial targeting away from probabilistic targeting and instead toward a precise access to distinct, yet complementary, networks (default mode, salience and executive control). Bayesian optimization was used to tailor stimulation parameters to each patient's preferences.

Using PFM data addressed the complexities of treating severe TRD and optimizing PCS settings. By employing this neuroimaging technique, the key large-scale dysfunctional networks involved in the pathophysiology and symptomatology of depression, or other neurological conditions, can be reliably identified at the single patient level. This knowledge can then be applied to the placement of multiple cortical paddles, allowing for a simultaneous and chronic modulation of these complementary networks. Additionally, Bayesian optimization can be employed to finetune the stimulation parameters. This method allows for the individual selection of the most effective parameters for each patient, ensuring a personalized and optimized clinical experience. Furthermore, given the high costs associated with randomized clinical trials, adaptive study designs offer a more efficient alternative. These designs allow for adjustments during the study based on accumulated data, enabling researchers to demonstrate both efficacy and clinical utility in a more cost-effective manner. By integrating PFM-guided PCS with a personalized optimization of stimulation parameters and an adaptive study design, the field of depression treatment can make significant strides in improving patient outcomes.

Like vagal nerve stimulation (VNS) or deep brain stimulation (DBS), electrical cortical stimulation involves implanting a pacemaker-like generator with multi-contact stimulating paddles placed over specific cortical regions. It modulates local and sub-cortical networks depending on stimulation intensity, frequency, and duration. The individually adaptive optimization paradigm for tuning stimulation parameters described in the present disclosure can be applicable to DBS and/or other neurological conditions like obsessive compulsive disorder, dystonia, or pain.

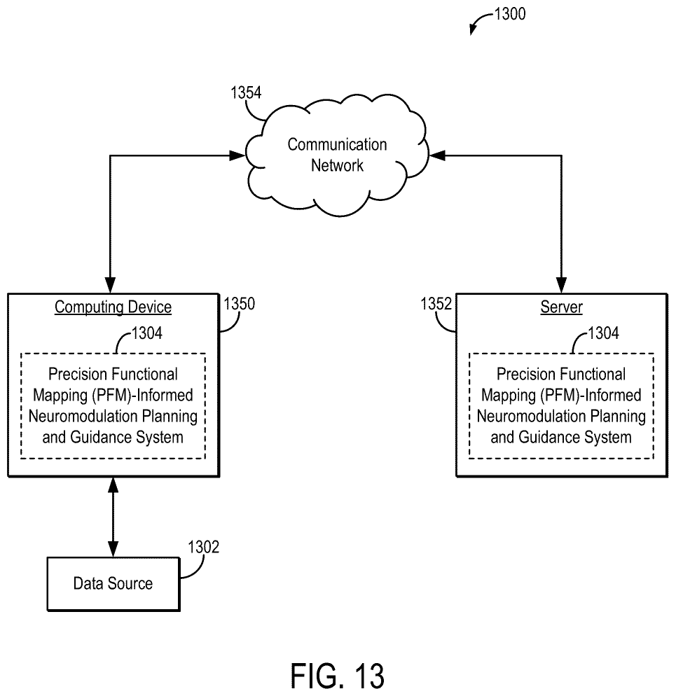

FIG. 13 shows an example of a system 1300 for generating precision functional mapping data and planning and/or guiding neuromodulation therapy based on those data in accordance with some embodiments described in the present disclosure. As shown in FIG. 13, a computing device 1350 can receive one or more types of data (e.g., magnetic resonance image data, time course signal data, precision functional mapping data) from data source 1302, which may be a magnetic resonance image source. In some embodiments, computing device 1350 can execute at least a portion of a precision functional mapping informed neuromodulation planning and guidance system 1304 to generate functional mapping data from data received from the data source 1302 and to guide the delivery of neuromodulation therapies based on those functional mapping data.

Additionally or alternatively, in some embodiments, the computing device 1350 can communicate information about data received from the data source 1302 to a server 1352 over a communication network 1354, which can execute at least a portion of the precision functional mapping informed neuromodulation planning and guidance system. In such embodiments, the server 1352 can return information to the computing device 1350 (and/or any other suitable computing device) indicative of an output of the precision functional mapping informed neuromodulation planning and guidance system.

In some embodiments, computing device 1350 and/or server 1352 can be any suitable computing device or combination of devices, such as a desktop computer, a laptop computer, a smartphone, a tablet computer, a wearable computer, a server computer, a virtual machine being executed by a physical computing device, and so on. The computing device 1350 and/or server 1352 can also reconstruct images from the data.

In some embodiments, data source 1302 can be any suitable source of image data (e.g., measurement data, images reconstructed from measurement data), such as an MRI system, another computing device (e.g., a server storing image data), and so on. In some embodiments, data source 1302 can be local to computing device 1350. For example, data source 1302 can be incorporated with computing device 1350 (e.g., computing device 1350 can be configured as part of a device for capturing, scanning, and/or storing images). As another example, data source 1302 can be connected to computing device 1350 by a cable, a direct wireless link, and so on. Additionally or alternatively, in some embodiments, data source 1302 can be located locally and/or remotely from computing device 1350, and can communicate data to computing device 1350 (and/or server 1352) via a communication network (e.g., communication network 1354).

In some embodiments, communication network 1354 can be any suitable communication network or combination of communication networks. For example, communication network 1354 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 1354 can be a local area network, a wide area network, a public network (e.g., the Internet), a private or semi-private network (e.g., a corporate or university intranet), any other suitable type of network, or any suitable combination of networks. Communications links shown in FIG. 13 can each be any suitable communications link or combination of communications links, such as wired links, fiber optic links, Wi-Fi links, Bluetooth links, cellular links, and so on.

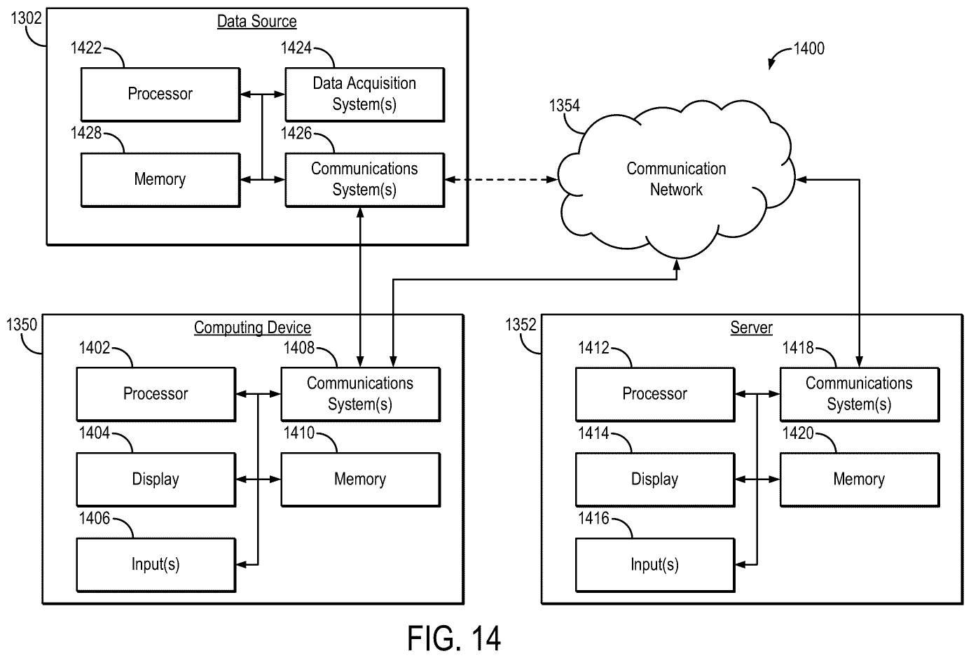

Referring now to FIG. 14, an example of hardware 1400 that can be used to implement data source 1302, computing device 1350, and server 1352 in accordance with some embodiments of the systems and methods described in the present disclosure is shown. As shown in FIG. 14, in some embodiments, computing device 1350 can include a processor 1402, a display 1404, one or more inputs 1406, one or more communication systems 1408, and/or memory 1410. In some embodiments, processor 1402 can be any suitable hardware processor or combination of processors, such as a central processing unit (“CPU”), a graphics processing unit (“GPU”), and so on. In some embodiments, display 1404 can include any suitable display devices, such as a computer monitor, a touchscreen, a television, and so on. In some embodiments, inputs 1406 can include any suitable input devices and/or sensors that can be used to receive user input, such as a keyboard, a mouse, a touchscreen, a microphone, and so on.

In some embodiments, communications systems 1408 can include any suitable hardware, firmware, and/or software for communicating information over communication network 1354 and/or any other suitable communication networks. For example, communications systems 1408 can include one or more transceivers, one or more communication chips and/or chip sets, and so on. In a more particular example, communications systems 1408 can include hardware, firmware and/or software that can be used to establish a Wi-Fi connection, a Bluetooth connection, a cellular connection, an Ethernet connection, and so on.

In some embodiments, memory 1410 can include any suitable storage device or devices that can be used to store instructions, values, data, or the like, that can be used, for example, by processor 1402 to present content using display 1404, to communicate with server 1352 via communications system(s) 1408, and so on. Memory 1410 can include any suitable volatile memory, non-volatile memory, storage, or any suitable combination thereof. For example, memory 1410 can include RAM, ROM, EEPROM, one or more flash drives, one or more hard disks, one or more solid state drives, one or more optical drives, and so on. In some embodiments, memory 1410 can have encoded thereon, or otherwise stored therein, a computer program for controlling operation of computing device 1350. In such embodiments, processor 1402 can execute at least a portion of the computer program to present content (e.g., images, user interfaces, graphics, tables), receive content from server 1352, transmit information to server 1352, and so on.

In some embodiments, server 1352 can include a processor 1412, a display 1414, one or more inputs 1416, one or more communications systems 1418, and/or memory 1420. In some embodiments, processor 1412 can be any suitable hardware processor or combination of processors, such as a CPU, a GPU, and so on. In some embodiments, display 1414 can include any suitable display devices, such as a computer monitor, a touchscreen, a television, and so on. In some embodiments, inputs 1416 can include any suitable input devices and/or sensors that can be used to receive user input, such as a keyboard, a mouse, a touchscreen, a microphone, and so on.

In some embodiments, communications systems 1418 can include any suitable hardware, firmware, and/or software for communicating information over communication network 1354 and/or any other suitable communication networks. For example, communications systems 1418 can include one or more transceivers, one or more communication chips and/or chip sets, and so on. In a more particular example, communications systems 1418 can include hardware, firmware and/or software that can be used to establish a Wi-Fi connection, a Bluetooth connection, a cellular connection, an Ethernet connection, and so on.

In some embodiments, memory 1420 can include any suitable storage device or devices that can be used to store instructions, values, data, or the like, that can be used, for example, by processor 1412 to present content using display 1414, to communicate with one or more computing devices 1350, and so on. Memory 1420 can include any suitable volatile memory, non-volatile memory, storage, or any suitable combination thereof. For example, memory 1420 can include RAM, ROM, EEPROM, one or more flash drives, one or more hard disks, one or more solid state drives, one or more optical drives, and so on. In some embodiments, memory 1420 can have encoded thereon a server program for controlling operation of server 1352. In such embodiments, processor 1412 can execute at least a portion of the server program to transmit information and/or content (e.g., data, images, a user interface) to one or more computing devices 1350, receive information and/or content from one or more computing devices 1350, receive instructions from one or more devices (e.g., a personal computer, a laptop computer, a tablet computer, a smartphone), and so on.

In some embodiments, data source 1302 can include a processor 1422, one or more inputs 1424, one or more communications systems 1426, and/or memory 1428. In some embodiments, processor 1422 can be any suitable hardware processor or combination of processors, such as a CPU, a GPU, and so on. In some embodiments, the one or more inputs 1424 are generally configured to acquire data, images, or both, and can include an MRI system. Additionally or alternatively, in some embodiments, one or more inputs 1424 can include any suitable hardware, firmware, and/or software for coupling to and/or controlling operations of an MRI system. In some embodiments, one or more portions of the one or more inputs 1424 can be removable and/or replaceable.

Note that, although not shown, data source 1302 can include any suitable inputs and/or outputs. For example, data source 1302 can include input devices and/or sensors that can be used to receive user input, such as a keyboard, a mouse, a touchscreen, a microphone, a trackpad, a trackball, and so on. As another example, data source 1302 can include any suitable display devices, such as a computer monitor, a touchscreen, a television, etc., one or more speakers, and so on.

In some embodiments, communications systems 1426 can include any suitable hardware, firmware, and/or software for communicating information to computing device 1350 (and, in some embodiments, over communication network 1354 and/or any other suitable communication networks). For example, communications systems 1426 can include one or more transceivers, one or more communication chips and/or chip sets, and so on. In a more particular example, communications systems 1426 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1428 can include any suitable storage device or devices that can be used to store instructions, values, data, or the like, that can be used, for example, by processor 1422 to control the one or more inputs 1424, and/or receive data from the one or more inputs 1424; to images from data; present content (e.g., images, a user interface) using a display; communicate with one or more computing devices 1350; and so on. Memory 1428 can include any suitable volatile memory, non-volatile memory, storage, or any suitable combination thereof. For example, memory 1428 can include RAM, ROM, EEPROM, one or more flash drives, one or more hard disks, one or more solid state drives, one or more optical drives, and so on. In some embodiments, memory 1428 can have encoded thereon, or otherwise stored therein, a program for controlling operation of data source 1302. In such embodiments, processor 1422 can execute at least a portion of the program to generate images, transmit information and/or content (e.g., data, images) to one or more computing devices 1350, receive information and/or content from one or more computing devices 1350, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for planning, guiding, or monitoring a delivery of neuromodulation to a subject's brain, the method comprising:
(a) accessing precision functional mapping data with a computer system;
(b) determining one or more target locations in the precision functional mapping data using the computer system, wherein the one or more target locations indicate locations to which neuromodulation should be delivered;
(c) generating target localization data by localizing the one or more target locations relative to the brain of the subject; and
(d) outputting target localization data with the computer system.

2. The method of claim 1, wherein outputting the target localization data comprises determining neuromodulation settings based on the one or more target locations and controlling a neuromodulation device to deliver neuromodulation to the one or more target locations according to the neuromodulation settings.

3. The method of claim 2, further comprising receiving feedback data from the subject in response to the neuromodulation settings and updating the neuromodulation settings based on the feedback data.

4. The method of claim 3, wherein the neuromodulation settings are updated using a Bayesian optimization framework.

5. The method of claim 4, wherein the computer system receives the feedback data from the subject and inputs the feedback data to the Bayesian optimization, wherein the feedback data indicate subject preference to different neuromodulation setting selections in the neuromodulation settings.

6. The method of claim 1, wherein outputting the target localization data comprises generating a report that indicates a neuromodulation treatment plan for delivering the neuromodulation to the one or more target locations.

7. The method of claim 6, wherein the neuromodulation treatment plan includes neuromodulation settings for delivering neuromodulation with a neuromodulation device to the one or more target locations.

8. The method of claim 1, wherein outputting the target localization data comprises generating a report that indicates an image-based guidance for delivering neuromodulation to the one or more target locations.

9. The method of claim 8, wherein generating the report comprises:
receiving medical image data of the subject with the computer system; and
generating composite image data by coregistering the target localization data with the medical image data.

10. The method of claim 9, wherein generating the composite image data further includes coregistering the precision functional mapping data with the target localization data and the medical image data.

11. The method of claim 1, wherein outputting the target localization data comprises generating a report that indicates monitoring delivery of neuromodulation to the one or more target locations.

12. The method of claim 1, wherein the precision functional mapping data comprise one or more individual-specific functional network maps of the subject's brain.

13. The method of claim 1, wherein the precision functional mapping data comprise one or more individual-specific integration zone maps of the subject's brain.

14. The method of claim 1, wherein the one or more target locations comprise brainordinates selected based on a condition for which neuromodulation therapy is being provided to the subject.

15. The method of claim 14, wherein the brainordinates comprise grayordinates.

16. The method of claim 14, wherein the brainordinates comprise voxels from one or more magnetic resonance images of the subject.

17. The method of claim 14, wherein the one or more target locations comprise clusters of brainordinates.

18. The method of claim 14, wherein determining the one or more target locations comprises determining a functional network that when modulated by a neuromodulation therapy provides a therapeutic effect to the subject and selecting brainordinates associated with the functional network.

19. The method of claim 14, wherein the condition comprises depression.
